(19) United States
(12) Patent Application Publication
(10) Pub. No.: US 2010/0198200 A1
(43) Pub. Date: Aug. 5, 2010

(54) Smart Illumination for Surgical Devices

(76) Inventor: Christopher Horvath, Irvine, CA (US)

Correspondence Address: ALCON, IP LEGAL, TB4-8, 6201 SOUTH FREEWAY, FORT WORTH, TX, 76134, US

(21) Appl. No.: 12/693,808

(22) Filed: Jan. 26, 2010

Related U.S. Application Data
(60) Provisional application No. 61/148,538, filed on Jan. 30, 2009.

(51) Int. Cl. A61B 17/00; A61B 18/20; A61F 9/007

(52) U.S. Cl. 606/10; 606/1

(57) ABSTRACT

In various embodiments, a device (such as a surgical footswitch, handpiece, etc.) and a surgical console may be used by an operator during a surgical procedure and/or in an office. The device may communicate with the surgical console to receive information associated with a procedure (e.g., information relative to the device, console, or other equipment used in a surgical procedure). In some embodiments, the information may be determined/generated at the device (i.e. and not necessarily received from the surgical console). Based on the information, a configuration may be determined (e.g., at the device, at the console, etc.) for one or more indicators on the device to provide at least part of the information to the operator of the device. The at least one indicator on the device may thus be illuminated to provide the operator at least part of the information associated with the procedure.

### PRIORITY

This application claims the benefit of priority of U.S. Provisional Patent Application Ser. No. 61/148,538 titled “Smart Illumination for Surgical Devices”, filed on Jan. 30, 2009, whose inventor is Christopher Horvath, which is hereby incorporated by reference in its entirety as though fully and completely set forth herein.

### FIELD OF THE INVENTION

The present invention generally pertains to surgical devices. More particularly, but not by way of limitation, the present invention pertains to indicators for surgical devices.

### DESCRIPTION OF THE RELATED ART

Surgeons may need to stay focused on a patient during a surgical procedure while at the same time also handle a multitude of tasks and surgical equipment. It may be necessary for a surgeon to review various parameters, statuses, etc. of surgical equipment during the surgical procedure. Often, the surgical console that displays this information is placed far away or even behind the surgeon making it difficult for the surgeon to review this information and stay focused on the patient.

During the use of a complex patient treatment apparatus or surgical system (such as surgical equipment used in performing ophthalmic surgery), the control of a variety of different subsystems, such as pneumatic and electronically driven subsystems may be required. The operation of the subsystems may be controlled by a microprocessor-driven console. The microprocessor controls within a surgical console may receive mechanical inputs from either the operator of the surgical system or from an assistant. A control input device, such as a footswitch, may be used to accept mechanical inputs. These mechanical inputs may originate from a movement of a foot of an operator to govern the operation of a subsystem within the patient treatment apparatus. The mechanical inputs from the movement of the foot of the operator may be translated into electrical signals that are fed to the microprocessor controls. The electrical signals may then be used to control the operational characteristics of a subsystem in a complex patient treatment apparatus.

Examples of footswitches that are designed for receiving mechanical inputs from the movement of the foot of an operator of a complex patient treatment apparatus may be found in several U.S. patents, including U.S. Pat. Nos. 4,837,857 (Scheller, et al.), 4,965,417 (Massie), 4,983,901 (Lehmer), 5,091,656 (Gahn), 5,268,624 (Zanger), 5,554,894 (Sepielli), 5,580,347 5 (Reimels), 5,635,777 (Telymonde, et al), 5,787,760 (Thorlakson), 5,983,749 (Holtorf), and 6,179,829 B1 (Bisch, et al), and in International Patent Application Publication Nos. WO 98/08442 (Bisch, et al.), WO 00/12037 (Chen), and WO 02/01310 (Chen). These patents and patent applications focus primarily on footswitches that include a foot pedal or tillable treadle similar to the accelerator pedal used to govern the speed of an automobile. The movement of the foot pedal or tillable treadle typically provides a linear control input. Such linear control inputs may be used, for example, for regulating vacuum, rotational speed, power, or reciprocal motion.

In more complex footswitch assemblies, side or wing switches may be added to housings on either side of the foot pedal in order to provide additional capabilities to the footswitch. The condition of these side or wing switches may be changed by the application of pressure from the front portion of the operator's foot or from the rear portion of the operator's foot. Further, in the prior art, footswitches for the operation of surgical lasers typically include a shroud to prevent inadvertent or accidental firing of a laser in the ready position.

### SUMMARY OF THE INVENTION

In various embodiments, a device (such as a surgical footswitch, handpiece, etc.) and a surgical console may be used by an operator during a surgical procedure (such as an ophthalmic surgical procedure) and/or in an office (e.g., for examination of a patient, to calibrate the device, etc). The device may communicate with the surgical console through an interface, for example, to receive information associated with the procedure (e.g., information relative to the device, console, or other equipment used in the procedure). In some embodiments, the information may be determined/generated at the device (i.e. and not necessarily received from the surgical console). Based on the information, a configuration may be determined (e.g., at the device, at the console, etc.) for one or more indicators on the device to provide at least part of the information to the operator of the device. A control signal may be generated to illuminate the at least one indicator according to the determined configuration. The at least one indicator on the device may thus be illuminated according to the communicated control signal to provide the operator at least part of the information associated with the procedure (e.g., surgical procedure, examination procedure, calibration, etc).

### BRIEF DESCRIPTION OF THE DRAWINGS

For a more complete understanding of the present invention, reference is made to the following description taken in conjunction with the accompanying drawings in which:

FIG. 1 illustrates a footswitch with a first arrangement of indicators, according to an embodiment;

FIG. 2 illustrates a footswitch with a second arrangement of indicators, according to an embodiment;

FIG. 3 illustrates a footswitch with a third arrangement of indicators, according to an embodiment;

FIG. 4a illustrates a footswitch with a fourth arrangement of indicators, according to an embodiment;

FIG. 4b illustrates a cross sectional view of the footswitch shown in FIG. 4a, according to an embodiment;

FIG. 4c illustrates a first functional diagram that illustrates a footswitch communicably coupled to a surgical system, according to an embodiment;

FIG. 4d illustrates a second functional diagram of a footswitch, according to an embodiment;

FIG. 4e illustrates a third functional diagram of a footswitch, according to an embodiment;

FIG. 4f illustrates a fourth functional diagram of a footswitch, according to an embodiment;

FIG. 4g illustrates a logic flow diagram illustrating a method of controlling surgical equipment, according to an embodiment;

FIG. 5 illustrates a footswitch with a fifth arrangement of indicators, according to an embodiment;

FIG. 6a illustrates a footswitch with a shroud and a sixth arrangement of indicators, according to an embodiment;

FIG. 6b illustrates diagram of another footswitch with a shroud and a seventh arrangement of indicators, according to an embodiment;

FIG. 6c illustrates a functional diagram of an embodiment of the multifunction surgical footswitch having a communication interface;

FIG. 7 illustrates a surgical console, according to an embodiment;

FIG. 8a illustrates a laser console and a laser indirect opthalmoscope (LIO), according to an embodiment;

FIG. 8b illustrates a side view of the LIO with indicators, according to an embodiment;

FIG. 8c illustrates a slit-lamp with doctor filter, according to an embodiment.

FIG. 9 illustrates a vitrectomy probe with indicators, according to an embodiment;

FIG. 10 illustrates a pneumatic handle with indicators, according to an embodiment;

FIG. 11 illustrates a torsional handpiece with indicators, according to an embodiment;

FIG. 12 illustrates an ultrasound handpiece with indicators, according to an embodiment;

FIG. 13 illustrates another ultrasound handpiece with indicators, according to an embodiment;

FIG. 14 illustrates a fragmentation handpiece with indicators, according to an embodiment;

FIG. 15 illustrates a diathermy/coagulation handpiece with indicators, according to an embodiment; and

FIG. 16 illustrates a flowchart of a method for providing information about parameters, console statuses, etc. to an operator through the use of indicators, according to an embodiment.

It is to be understood that both the foregoing general description and the following detailed description are exemplary and explanatory only and are intended to provide a further explanation of the present invention as claimed.

### DETAILED DESCRIPTION OF THE EMBODIMENTS

### Incorporation by Reference

U.S. patent application Publication entitled “Footswitch Operable to Control a Surgical System,” Publication No. 20060219049, Ser. No. 11/389,808, by Christopher Horvath, Mark Buczek, and T. Scott Rowe filed Mar. 27, 2006 is hereby incorporated by reference in its entirety.

U.S. patent application Publication entitled “Multifunction Surgical Footswitch,” Publication No. 20070043339, Ser. No. 11/474,668, by Christopher Horvath and Bruno Dacquay filed Jun. 26, 2006 is hereby incorporated by reference in its entirety.

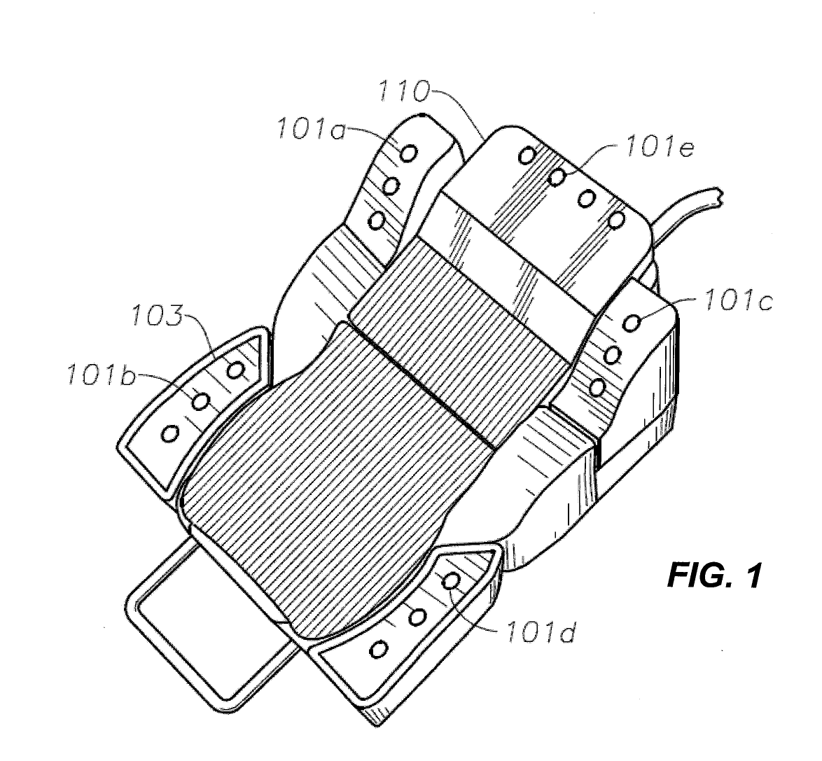

FIG. 1 illustrates a footswitch 110 with a first arrangement of indicators, according to an embodiment. Indicators (such as indicators 101a-e) may be placed on surgical devices that are controlled by an operator (e.g., footswitches, handpieces, other adaptations, etc.) and/or other surgical devices that are in view of and/or in close proximity to the operator. In some embodiments, operators may control surgical devices by physically engaging (through holding, applying pressure to, etc.) the surgical devices. Indicators may also be placed on surgical devices controlled by and/or in view of surgical staff, the patient, etc. Indicators may provide the operator/staff with information associated with a procedure (e.g., surgical procedure, examination procedure, calibration, etc.) and/or the equipment used in the procedure. For example, information provided by the indicators may include surgical parameters and/or the status of a surgical console or other surgical device (such as footswitches, handpieces, other adaptations, etc). For example, status information may include (e.g., “Ready”, “Stand-by”, “System Error”, etc). As another example, indicators on a multi-function footswitch/handpiece may illuminate in various colors and/or positions to indicate a current configuration programmed for the footswitch/handpiece.

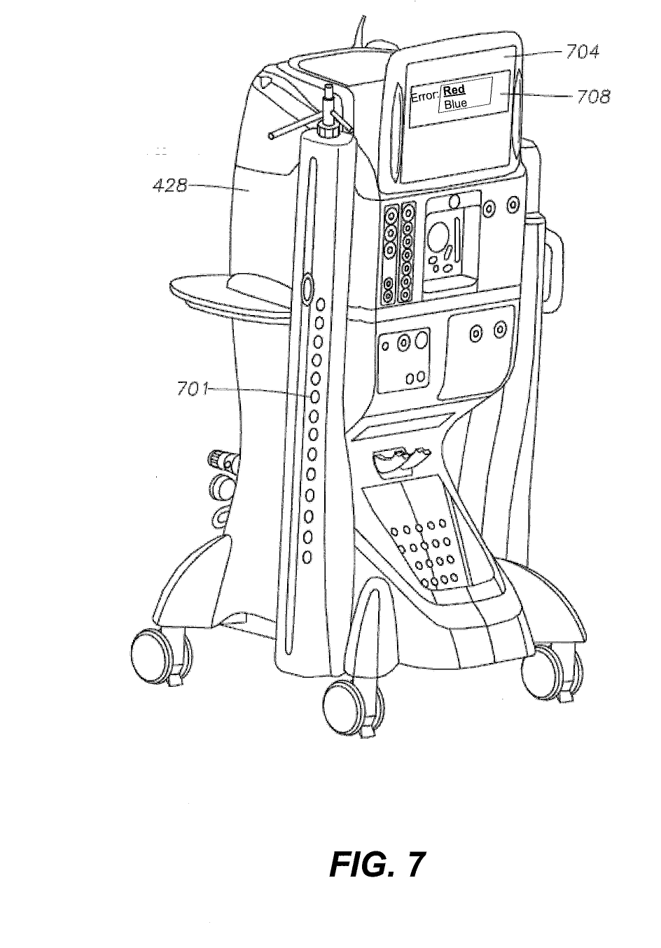

Indicators may include light emitting diodes (LEDs) (such as organic LEDs (OLEDs), polymer LEDs (PLEDs), solid-state lighting (SSL), etc.), optical fibers, incandescent light sources (e.g., a light bulb), electroluminescent wires/sheets, etc. Other types of indicators may also be used. Indicators may be illuminated according to one or more characteristics that may be used to provide information (e.g., information associated with a surgical procedure and/or the equipment used in the surgical procedure). Characteristics may include, for example, illuminated indicator position, illumination color, illumination pattern, etc. In some embodiments, the illuminated indicator position, illumination color, illumination pattern, etc. may be provided to an operator along with the associated characteristics/information through a user manual, web page, etc. In some embodiments, the operator may establish (e.g., through a user interface 708 shown in FIG. 7) the associations between the characteristics/information and the illuminated indicator position, illumination color, illumination pattern, etc. For example, the operator may select “red” as the color to use for the illuminators when an error is detected. In some embodiments, the user interface may be displayed (e.g., with drop down menus, editable fields, check boxes, etc.) on screen 704 (see FIG. 7).

In some embodiments, illuminated indicator positions on footswitch 110 may indicate which port of a dual port laser (controlled by the footswitch 110) is active. For example, indicators on the left side (e.g., indicators 101a-b) may be illuminated to indicate a left laser port is active while indicators on the right side (e.g., indicators 101c-d) may be illuminated when the right laser port is active. The indicators may also be illuminated to indicate when a button or portion of the footswitch is pressed (e.g., indicators 101b may be illuminated when button 103 is pressed on footswitch 110). In some embodiments, the indicators may also provide illumination for the device to make the device easier to see and use in a dark environment.

As another example, illumination color may be used to indicate console status (e.g., indicators 101e may illuminate green to indicate the console is “Ready”, white to indicate the console is in “Stand-by”, or orange to indicate an error has been detected). In some embodiments, separate indicators may present separate colors or multiple indicators (e.g., all of the indicators) may be illuminated a specific color to convey status information.

In some embodiments, indicators may also provide an illumination pattern (e.g., a timed series of blinks) to illustrate a surgical sequence. For example, indicators 101e may blink in a pattern that is the same as the pulse pattern of an ultrasonic handpiece or the same as a laser pattern for a laser handpiece. The blinking pattern may be displayed prior to application to allow the operator (and/or staff) to adjust the pattern and/or may be provided during the actual firing sequence to provide confirmation of the firing sequence to the operator and/or staff.

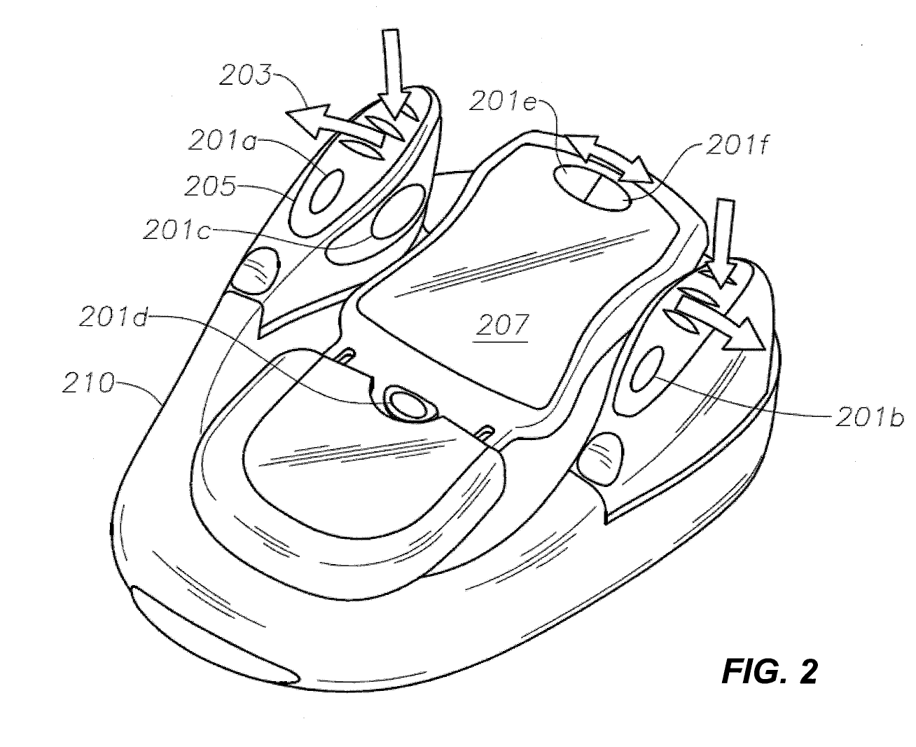

FIG. 2 illustrates a footswitch with a second arrangement of indicators, according to an embodiment. Indicators may include various shapes and sizes. For example, indicators may be circular, square, rectangular, oval, triangular, etc. As seen in FIG. 2, indicators 201a-f on footswitch 210 may be used to indicate different motions of switches/buttons on the footswitch. Arrows (e.g., arrow 203) are shown in FIG. 2 to illustrate some examples of possible button/switch movement on footswitch 210. In some embodiments, indicator 201c (and/or indicator 201a) may be illuminated when an operator pushes switch 205 in the direction of arrow 203. As another example, indicator 201e may illuminate when central portion 207 is moved to the left and indicator 201f may illuminate when central portion 207 is moved to the right. In some embodiments, indicators 201e,f may illuminate together to show a firing sequence. In some embodiments, the indicators may illuminate under an operator's foot and, although not directly visible, may provide light that is visible on the side of the operator's foot. Indicators 201a,b may be illuminated based on an active port of a dual port laser (e.g., indicator 201a may be illuminated to indicate the left laser port is active).

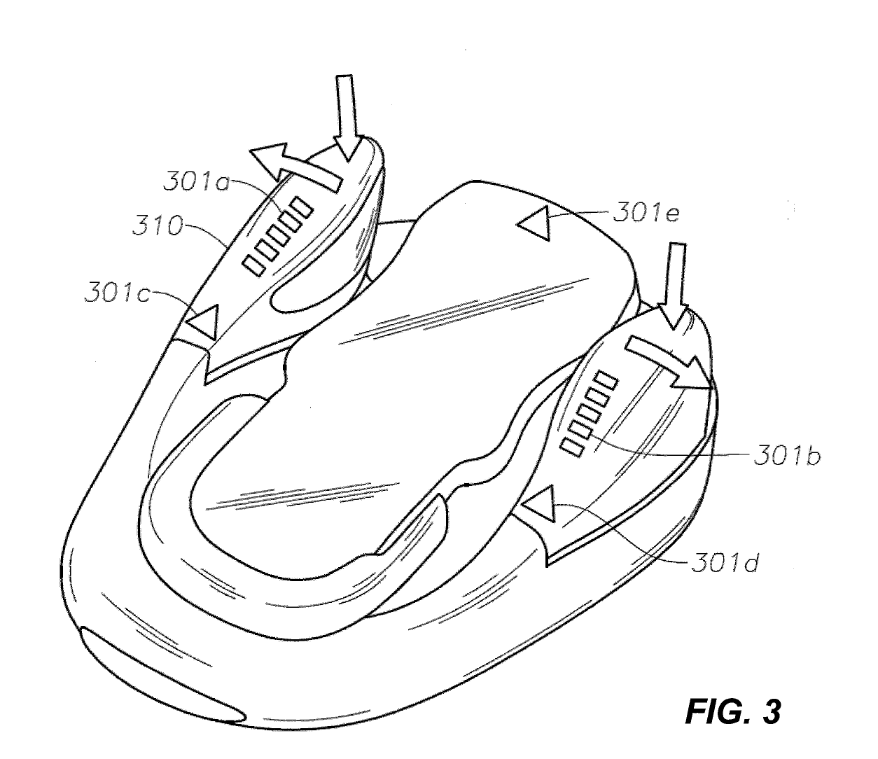

FIG. 3 illustrates a footswitch 310 with a third arrangement of indicators, according to an embodiment. In FIG. 3, a series of rectangular indicators 301a,b are shown. The indicators may be illuminated in sequence based on an amount of power being applied (e.g., one indicator in the array of indicators 301a may be illuminated (as the footswitch is pressed) to indicate low power, three indicators to indicate medium power and all five indicators of indicators 301a may be illuminated to indicate full power). In some embodiments, the indicators 301a,b may be illuminated relative to which port of a dual port laser is active. For example, indicators 301b may be illuminated when the right laser port is active (and, further, only three of these indicators 301b may be illuminated when the right laser port is active with medium power). In some embodiments, triangular indicators 301c-e may be illuminated with a firing sequence and/or console status information. In some embodiments, the firing sequence and console status information may be presented together (e.g., indicator 301e may blink in a similar timed sequence as the programmed firing sequence and may blink in a green color to indicate the console is “Ready”). Other colors and patterns are also contemplated (e.g., indicators 301a-e may all illuminate in a continuous red color to indicate a system error).

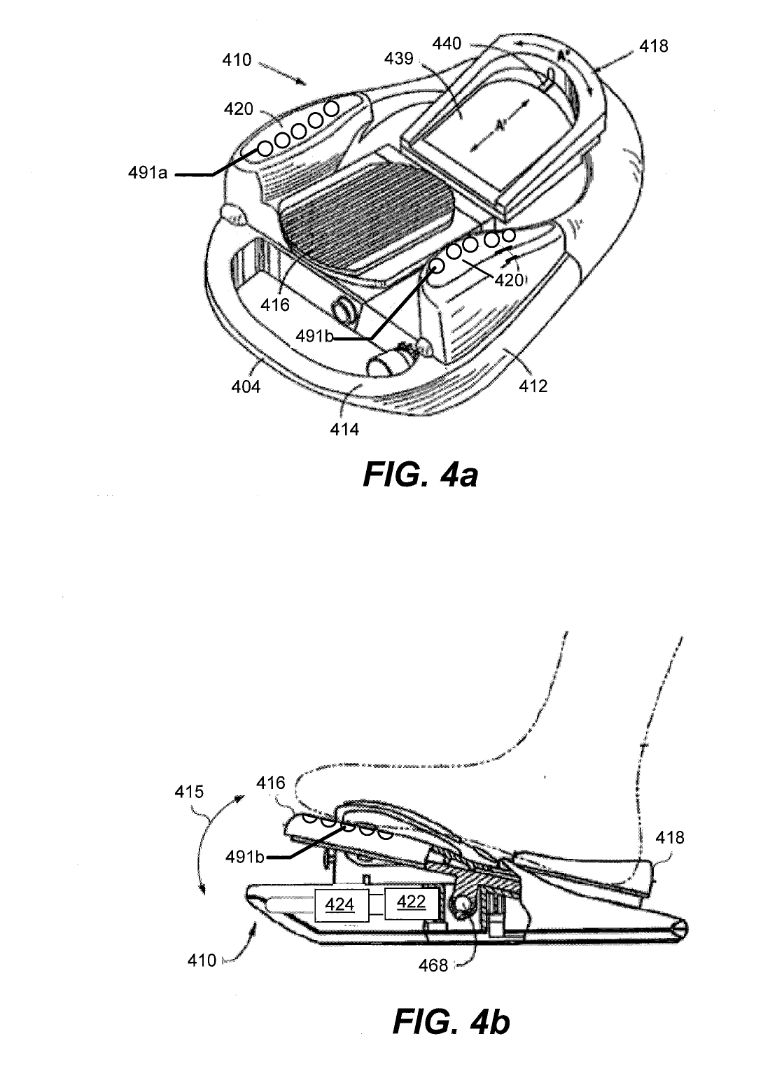

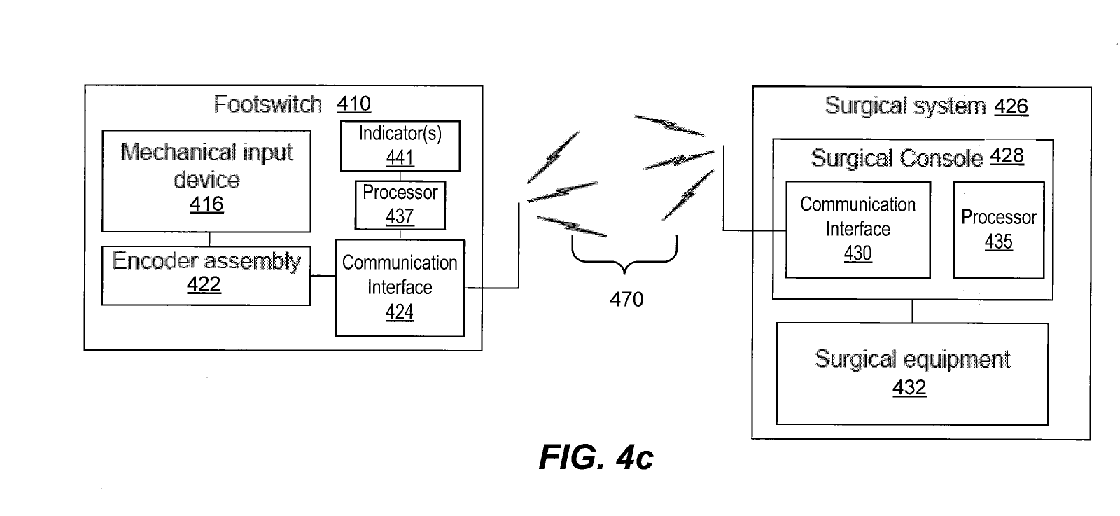

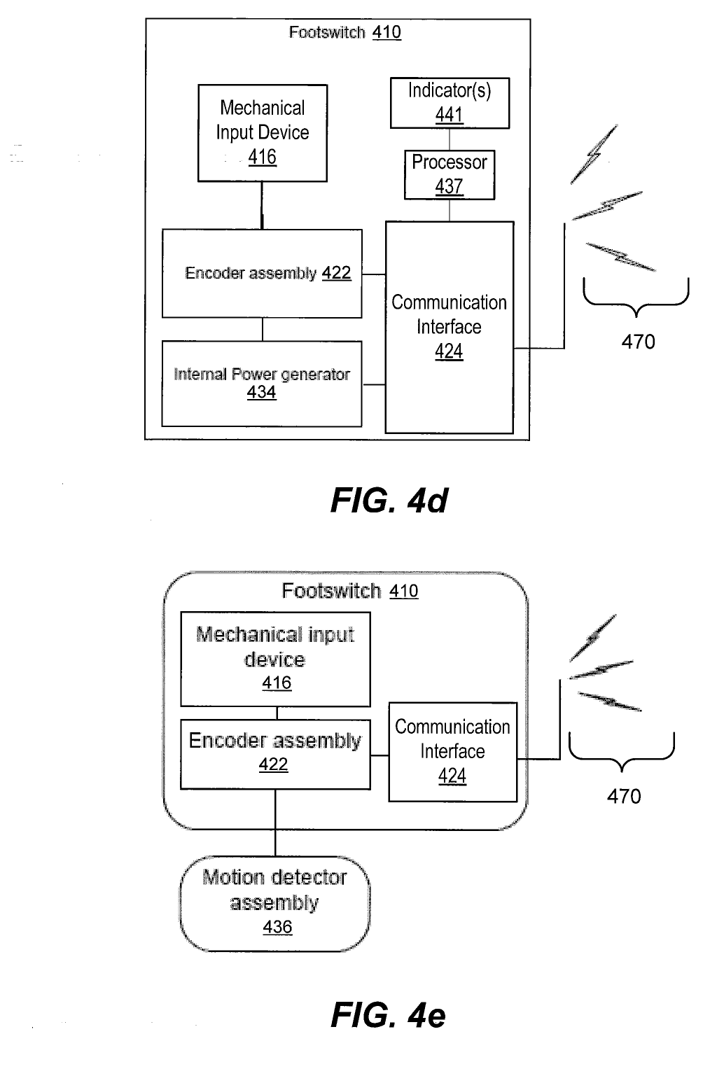

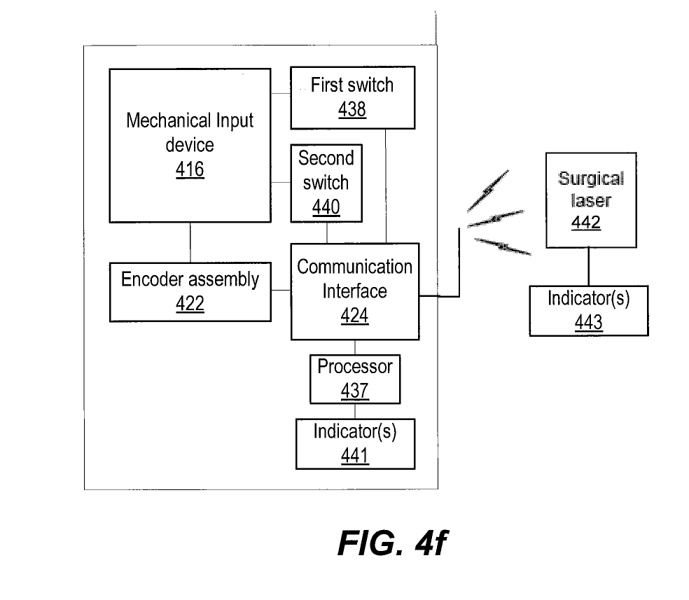

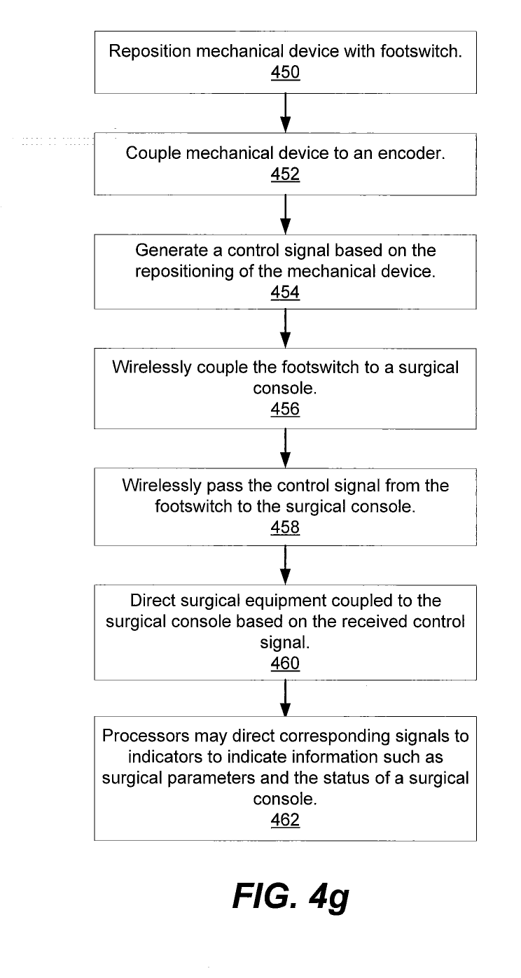

FIG. 4a illustrates a footswitch 410 with a fourth arrangement of indicators, according to an embodiment. The footswitch 410 may include a body or housing that further includes bottom housing 412 and top housing 414, and a foot pedal or treadle 416, all of which may be made from a material such as stainless steel, titanium or plastic. Embodiments may additionally include a separate heel cup assembly 418 (e.g., with section 439) and a handle 404 positioned in the front. Side or wing switches 420 may be placed on the top of housing 414 on either side of the foot pedal 416. Indicators 491a-b may also be positioned on the footswitch 410.

In some embodiments, an encoder assembly 422, as illustrated in the cross section illustrated in FIG. 4b, may be attached to the foot pedal or tillable treadle 416. Encoder assembly 422 may translate an angular or pitch position of the foot pedal or treadle 416, which may be tillable with respect to a horizontal plane or to a neutral or home plane, from a mechanical input based on the movement of the operator's foot into an electrical signal. Thus, the pitch 415 movement of the foot pedal or tillable treadle 416, which may be in a downward direction, may provide a control input. The control input may include a linear control input. In some embodiments, when a variable high input and a constant low input is satisfactory, the neutral or home plane may provide the constant low input, and depression of the foot pedal may be used for the variable high input.

FIG. 4c illustrates a first functional diagram of the footswitch 410 communicably coupled to a surgical system 426 (e.g., through pathway 470 which may be a wireless pathway and/or a physical (e.g., cabled) pathway). Footswitch 410 may include a mechanical input device such as pedal 416 that couples to encoder assembly 422 to produce a control signal that is provided to communication interface 424 (which may provide a wired or wireless interface). Communication interface 424 may be operable to provide wired communications (e.g., through a cable between the footswitch 410 and the console 428) or wireless communications between footswitch 410 and surgical system 426. In some embodiments, communication interface 424 may communicatively couple to communication interface 430 (which may be a wired or wireless interface) of surgical console 428. Thus, the control signal(s) produced by encoder assembly 422 may be communicated to surgical console 428 via a wired or wireless pathway 470. Surgical console 428 may be operable to direct surgical equipment 432 based on the control signal(s) that are relayed from the footswitch to the surgical console.

In some embodiments, surgical console 428 may also determine (e.g., using processor 435) one or more signals to control one or more indicators 441. In some embodiments, signals to control the indicators 441 (e.g., indicators 101a-e, 201a-f, 301a-e, etc.) may be communicated between communication interfaces 424 and 430. In some embodiments, a processor 437 may receive the signals and control the indicators 441. In some embodiments, signals may control the indicators 441 without processor 437 (e.g., the signals may directly power the corresponding indicators 441). The processors 435,437 may include embedded memory or may be coupled to a memory configured to store program instructions executable by the processor 435,437 to control the indicators for providing information to the operator/staff.

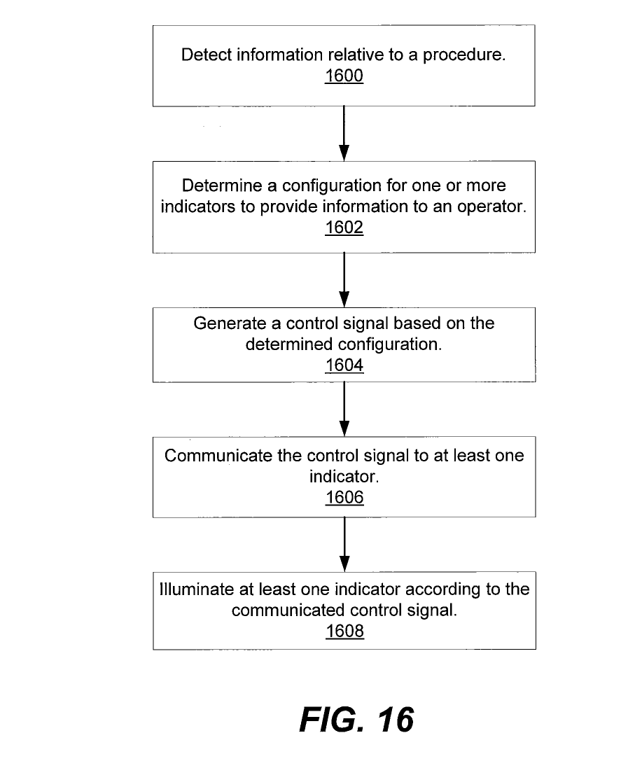

The processors 435,437 may include single processing devices or a plurality of processing devices. Such a processing device may be a microprocessor, micro-controller, digital signal processor, microcomputer, central processing unit, field programmable gate array, programmable logic device, state machine, logic circuitry, control circuitry, analog circuitry, digital circuitry, and/or any device that manipulates signals (analog and/or digital) based on operational instructions. The memory coupled to and/or embedded in the processors 435,437 may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processors 435,437 implement one or more of their functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. For example, the memory may store, and one or more processors 435/437 may execute, operational instructions corresponding to at least some of the elements illustrated and described in association with FIG. 4g and FIG. 16.

FIG. 4d illustrates a second functional diagram of a footswitch 410, according to an embodiment. Footswitch 410 may include a mechanical input device such as pedal 416 that couples to encoder assembly 422 to provide a control signal to a surgical console 428 via a wired or wireless communication pathway established through communication interface 424. The embodiment of FIG. 4d further includes an internal power generator 434 operable to translate movement of footswitch 410 into stored energy operable to be used to power and operate the encoder assembly 422, communication interface 424, and other components within footswitch 410 such as processor 437 and indicators 441. Internal power generator 434 may both generate and store energy with which to operate footswitch 410, power indicators 441, power processor 437, etc. This may eliminate potential failure of the footswitch 410 during a procedure and overcome the need to replace batteries within the footswitch 410. There are many different ways to derive power from the movement of the surgical footswitch 410. These include, for example, the piezoelectric effect, inductive power generation, the compression storage of compressed fluids (such as air and mechanical flywheels), etc. For example, when the piezoelectric effect is used to generate and store electrical energy, the mechanical energy provided by the operator to depress the pedal may compress a piezoelectric material that generates a voltage based on the mechanical energy exerted on the piezoelectric material. This electrical energy may then be stored within a capacitor or rechargeable battery to provide a power reserve within the footswitch 410. In another embodiment, the internal power generator 434 may use inductive power generation wherein movement of the footswitch may produce results in relative motion between an internal magnet and a series of coils to charge a capacitor or rechargeable battery. Energy may also be stored in the form of mechanical energy wherein the pedal is used to spin a flywheel, which in essence is a mechanical battery. Flywheels store energy mechanically in the form of kinetic energy. Alternatively, air or other fluids can be compressed and stored and then this compressed air may be used to generate energy to power footswitch 410. These are just examples of how internal power generator 434 may generate and store energy within the footswitch 410.

Processor 435 or processor 437 may prompt the operator to charge the footswitch should the stored energy within internal power generator 434 fall below a pre-determined level. For example, signals from processor 435, 437 may cause corresponding indicators 441 (e.g., one or more indicators 491a-b) to illuminate to indicate the low power (e.g., according to an indicator position next to a written “Low Power” designator on the footswitch or a color of the indicators (e.g., the indicators 491a-b on the footswitch 410 may blink yellow when power is low). Other indicator configurations are also contemplated. Alternatively, the surgical console 428 (e.g., processor 435) may direct the operator to charge the footswitch 410 should the stored energy fall below a pre-determined level. In some embodiments, one or more indicators 491a,b may illuminate a green color to indicate that the footswitch 410 is powered and ready for use.

The ability to power the footswitch 410 based on motion of the footswitch 410 or the mechanical motion provided by the operator may eliminate the need for batteries. In some embodiments, the footswitch 410 may prompt the operator (e.g., through the use of indicators 441) to recharge the footswitch 410 prior to the power falling below a pre-determined level (e.g., by blinking yellow when power is low). This may help ensure conditions where communications between the footswitch 410 and a surgical console 428 are interrupted by power failures in the footswitch 410 that can result in improper control signals that have the potential to injure a patient. Additionally, guidelines or processes may be established and implemented by the processors 435,437 such that should the wired/wireless communications between the footswitch 410 and surgical console 428 fail, the surgical equipment (e.g., surgical equipment 432) may return to a pre-determined position or mode of operation to prevent potential injury of a patient.

Returning to FIG. 4a, footswitch 410 may provide additional proportional control inputs utilizing heel cup assembly 418 which may enable an arcuate movement. In some embodiments, the heel cup assembly 418 may be positioned at the rear portion of the footswitch 410 to engage the heel of the operator. The heel cup assembly 418 may allow the operator to rotate the heel cup assembly 418 through an arcuate path while the operator's heel effectively remains in the same spot with respect to the footswitch 410. This angular position mechanical input to a potentiometer 468 may produce an electrical signal received by encoder assembly 422. This electrical signal may be an additional control signal from the footswitch 410 to the surgical system 426. This control signal may be either linear or non-linear. The electrical signal may also be received by processors 435,437 to use in determining corresponding signals for indicators 441. For example, one or more indicators 441 may indicate a current power or a current change in power being applied through the footswitch 410.

To further enhance operator control, an on/off switch may be included in the heel cup assembly 418 to activate a signal output from the potentiometer 468. Alternatively, such on/off switches may also be used to prevent inadvertent activation of the side switches 420. Such an on/off switch may be a slide switch moving along a linear path within the heel cup assembly 418, as is designated by the arrow marked ‘A’ illustrated in FIG. 4a. In some embodiments, indicators 441 may be used to indicate a status of the on/off switch (e.g., one or more of indicators 491a,b may be an LED that is illuminated in a green color when the switch is in the “On” position).

FIG. 4e illustrates a third functional diagram of a footswitch 410, according to an embodiment. For example, motion detector assembly 436, which may be powered by a cable or internal battery, or self-powered as discussed previously with respect to internal power generator 434, within footswitch 410 may be worn by an operator. Motion detector assembly 436 may transmit motion information to the surgical footswitch 410 and/or surgical console 428. The surgical footswitch 410 and/or surgical console 428 may receive the motion information and may produce additional signal(s) (e.g., control signals, indicator signals, etc.) based on the received information. The motion detector assembly 436 may be tethered and physically connected to footswitch 410 or wirelessly coupled to footswitch 410. In some embodiments, the motion detector assembly 436 may include one or more indicators (e.g., to indicate when motion is detected, a status of the surgical console 428, etc).

The operator may wear motion detector assembly 436 on a body part such as the knee, foot, arm, waist, head, fingers, shoulder, etc. Motion detector assembly 436 may transmit information (e.g., position/motion information, which may take the form of relative position information with respect to footswitch 410) to the surgical footswitch 410. This information may then be passed to a surgical console 428 and may be used as a one, two, or three dimensional linear switch. Motion detector assembly 436, in combination with footswitch 410, may enhance the control capability up to four or more independent dimensions. The encoder assembly 422 may then generate additional control signals based on the received motion information.

The localization of the motion detector assembly 436 may be performed through many distinct methods. For example, acceleration sensors may be incorporated within the motion detector assembly 436 wherein the acceleration of the motion detector assembly 436 may be integrated over time to provide motion information. Another example may use radio triangulation through multiple received signals emitted within the surgical theater. This may include a passive means of determining the motion information associated with the motion detector assembly 436. Alternatively, a radio frequency emitter within the motion detector assembly 436 may produce signals that are received by various receivers coupled to either the surgical footswitch 410 or surgical console 428 wherein the footswitch 410 or console 428 may be operable to process this information to produce both motion information associated with the motion detector assembly 436 and a control signal resulting from the processing of the motion information. Motion information and/or the control signals resulting from the processing of the motion information may be further used to determine configurations for the indicators 441 or 443.

FIG. 4f illustrates a fourth functional diagram of a surgical footswitch 410, according to an embodiment. Here surgical footswitch 410 may include a mechanical input device, such as pedal 416, an encoder assembly 422, a communication interface 424, processor 437, and indicators 441. This embodiment further includes two switches that may mechanically couple to the mechanical input device 416, first switch 438 and second switch 440. First switch 438 activates a first control signal as pedal 416 orients past a first determined point. When first switch 438 is activated, a first control signal is produced that is operable to initialize, for example, surgical laser 442 within the surgical system 426. This first switch 438 may be activated when the pedal 416 is initially depressed. The second switch 440 may produce a second control signal offset in time from the first control signal produced by the activation of first switch 438. For example, second switch 440 may be activated as pedal 416 nears the end of its angular motion; i.e., when the pedal 416 is fully depressed. This second control signal may direct the firing of surgical laser 442. In some embodiments, the status of first switch 438 and/or second switch 440 may be relayed through indicators 441 or 443.

In some embodiments, a trigger time between the activation of first switch 438 and second switch 440 may allow stress on surgical laser 442 to be reduced as surgical laser 442 may not be ramped to power. The trigger time between the activation of first switch 438 and second switch 440 may allow surgical laser 442 to “slowly” warm up before firing. In one embodiment, the trigger time between the activation of the two switches 438/440 may be between about 100 milliseconds and 300 milliseconds. The actual time may depend on the foot speed of the operator. This may allow surgical laser 442 to be slowly ramped to power over a span of about 100 milliseconds to about 300 milliseconds (some lasers cannot be turned on in less than 50 milliseconds). In some embodiments, the current power status of the laser may be shown through indicators (e.g., indicators 301a,b which may light up in sequential order to relay a current power status of the laser and/or may blink to show the programmed firing sequence of the laser). The reduced stress associated with firing surgical laser 442 may result in an improved surgical laser 442 performance and reliability. Although footswitch 410 is illustrated in this embodiment as establishing a wireless communication pathway between the footswitch 410 and surgical laser 442, footswitch 410 may also physically couple to the control circuits associated with initializing and firing laser surgical 442. In some embodiments, laser 442 may further be coupled to indicators 443 which may also be used to show laser status/firing pattern, surgical console status, etc.

FIG. 4g illustrates a logic flow diagram illustrating an embodiment of a method for controlling surgical equipment and associated indicators. This method involves repositioning a mechanical device within, for example, a footswitch 410 at 450. While footswitch 410 is used with respect to FIG. 4g, the method of FIG. 4g also applies to other footswitches (e.g., footswitch 110, 210, 310, etc). Footswitch 410 may be powered by an internal power generator operable to translate footswitch movement into stored energy. This may allow footswitch 410 to be self powered and may eliminate the need to physically couple footswitch 410 to a surgical console 428 or to a power supply. Additionally, this may eliminate the potential hazards associated with power failures within footswitch 410 during a medical procedure. The repositioning of the pedal within the surgical footswitch 410 may provide mechanical energy that is translated and stored as energy to operate the footswitch. The repositioning of the pedal within the surgical footswitch 410 may also allow control signals to be generated based on the motion and positioning of the pedal. Additional switches or mechanical assemblies within footswitch 410 may also receive mechanical input that can be translated into control signals.

The pedal or mechanical device may couple to an encoder at 452. This may allow the encoder to generate control signal(s) based on the repositioning of the mechanical device or pedal at 454. The footswitch 410 may communicatively couple (e.g., through wired and/or wireless communications) to the surgical console at 456. This communicative coupling may facilitate the transfer of data and other information between footswitch 410 and surgical console 428. At 458, the control signal from footswitch 410 may be communicated (e.g., passed wirelessly) to surgical console 428. Surgical console 428, at 460, may be operable to direct surgical equipment coupled to console 428 based on the received control signals. At 462, processors 435/437 may direct corresponding signals to indicators 441 to indicate information such as surgical parameters and surgical console statuses.

In the embodiments where footswitch 410 includes an internal power generator 434, internal power generator 434 may translate footswitch movement into stored energy using processes such as an inductive power generation, piezoelectric power generation, or other like processes. This may eliminate potential hazards associated with power failures within the footswitch that may result in unexpected control signals that produce potentially hazardous situations during surgery that could endanger a patient. Communication between footswitch 410 and surgical console 428 may be monitored such that a communication failure may result in a processor 435 within surgical console 428 directing the surgical equipment to a safe condition to avoid potential harm to a patient. In some embodiments, the communication failure and/or safe condition may be indicated to an operator through indicators 441.

In some embodiments, surgical footswitch 410 may include a base, a pedal 416, an encoder assembly 422, a communication interface 424, and an internal power generator 434. The pedal 416 may mount upon the base and may pivot. The encoder assembly 422 may couple to pedal 416. As pedal 416 pivots, the encoder assembly 422 may translate the mechanical signal of pedal 416 into a control signal based on the pedal's position and/or orientation. The communication interface 424 may couple to the encoder assembly 422 to receive the control signal. The communication interface 424 may also couple surgical footswitch 410 to surgical console 428 operable to control and direct surgical equipment 432. The communication interface 424 may pass the control signal from the encoder assembly 422 to the surgical console 428, which may then direct the surgical equipment 432 based on the control signal. This communication interface 424 may be wireless to eliminate wires or tethers that may be a hazard during surgery. The internal power generator 434 may translate footswitch 410 movement into stored energy to eliminate potential failures of the footswitch 410 during a procedure and thus overcome the need to replace batteries within footswitch 410.

Other embodiments may include a dual switch surgical footswitch 410 operable to ramp and fire surgical laser 442. First switch 438 may couple to pedal 416 and may be activated as pedal 416 orients past a first predetermined point as pedal 416 is initially depressed. When first switch 438 is activated, a first control signal may initialize surgical laser 442 within surgical system 426. A second switch 440 may also operably couple to pedal 416 and may be activated when pedal 416 orients past a second predetermined point. This second control signal may direct the firing of ramped surgical laser 442. The trigger time between the activation of first switch 438 and second switch 440 may allow stress on surgical laser 442 to be relieved by allowing surgical laser 442 to be ramped to power. In some embodiments, the power level, firing pattern, and/or port (left port/right port) may be indicated through the indicators 441.

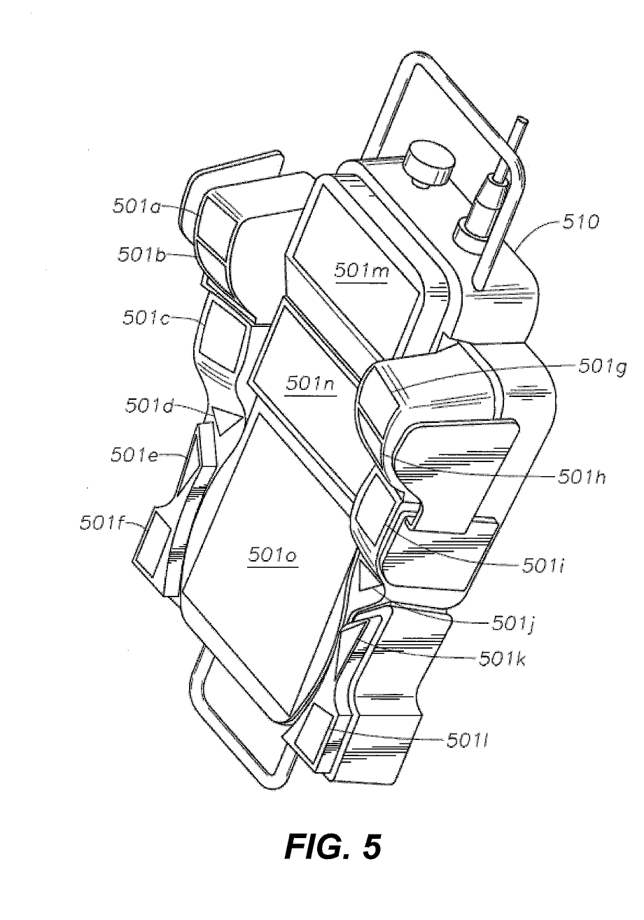

FIG. 5 illustrates a footswitch 510 with a fifth arrangement of indicators, according to an embodiment. In some embodiments, indicators (e.g., indicators 501a-o) may cover a large percentage of the visible area of a footswitch 510. In some embodiments, the indicators may be internal to the footswitch components and the various footswitch components may be transparent such that light from the indicators may be visible through the surface of the footswitch 510. Other configurations of the indicators are also contemplated.

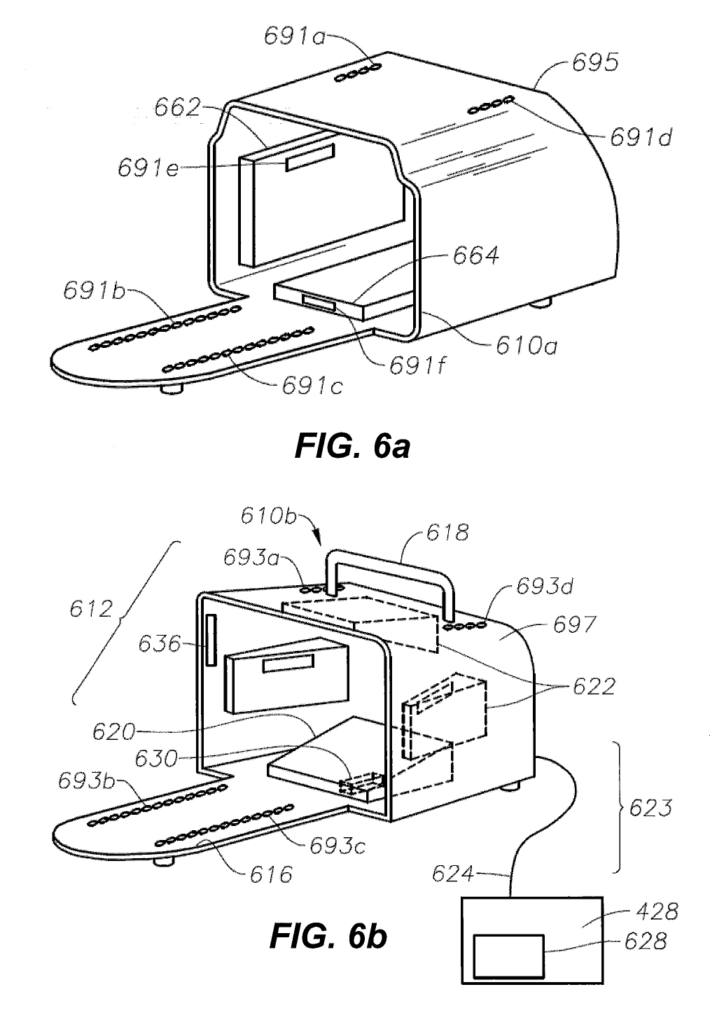

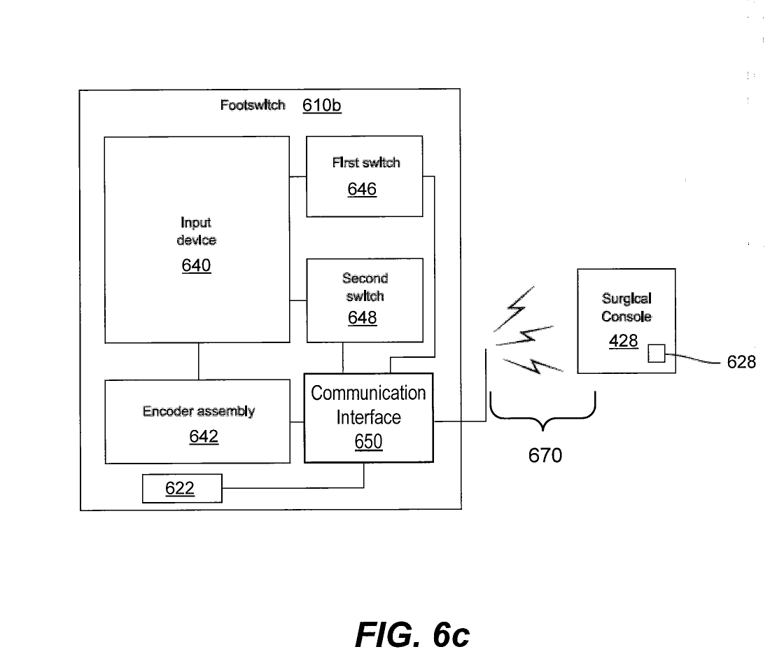

FIG. 6a illustrates a footswitch 610a with a shroud and a sixth arrangement of indicators, according to an embodiment. Various embodiments provide a multifunction surgical footswitch that allows an operator to both place a surgical laser in a ready condition and fire the laser once it is in the ready condition. Embodiments may include a multi-position switch or multiple switches for controlling various functions. For example, switches on the footswitch may allow an operator to control power, laser firing mode (e.g., “Ready” and “Stand-by”), etc.

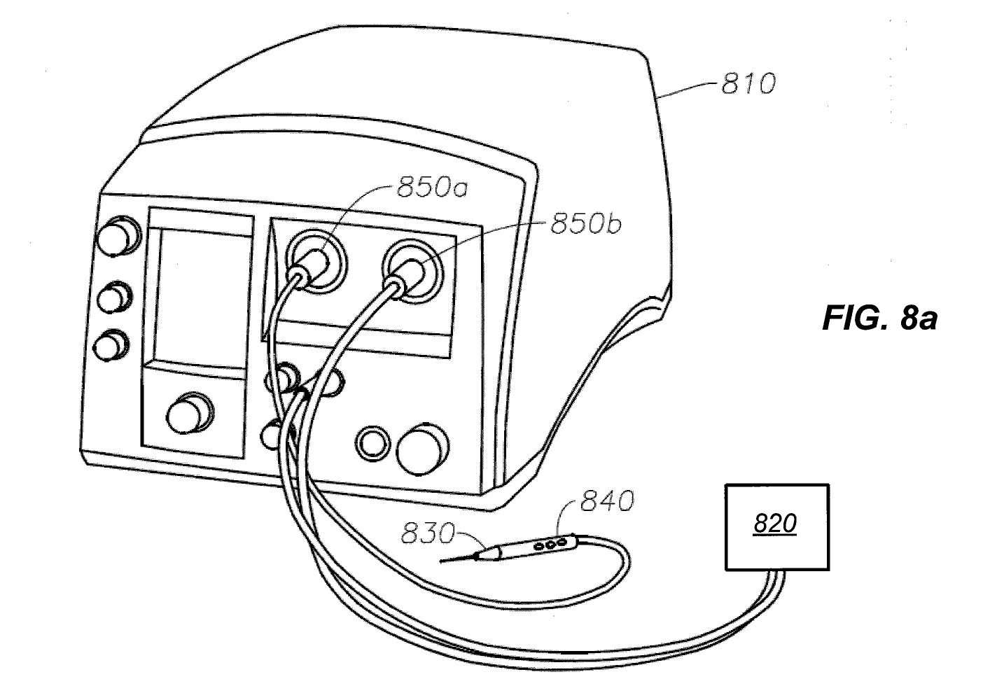

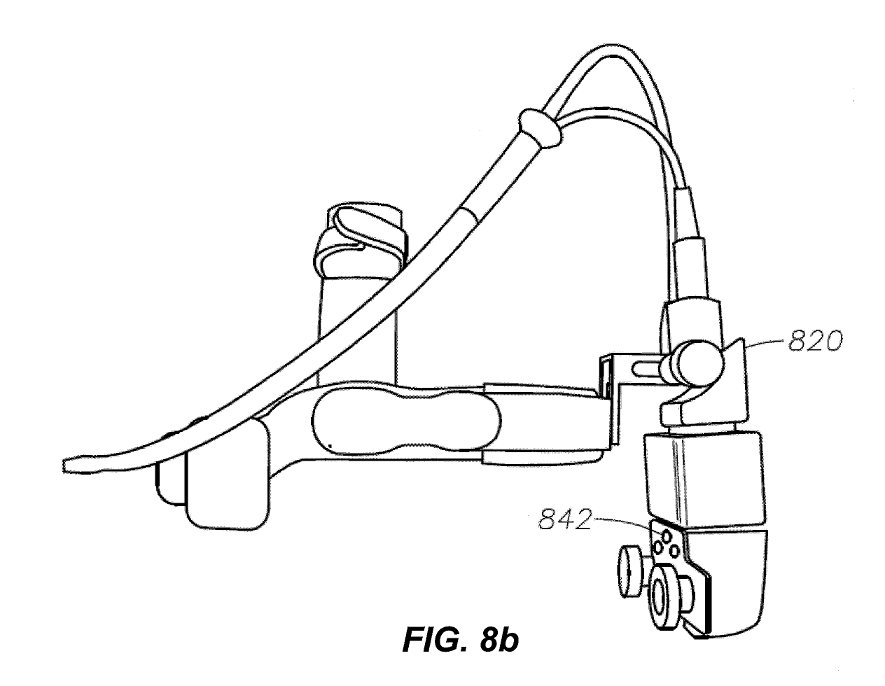

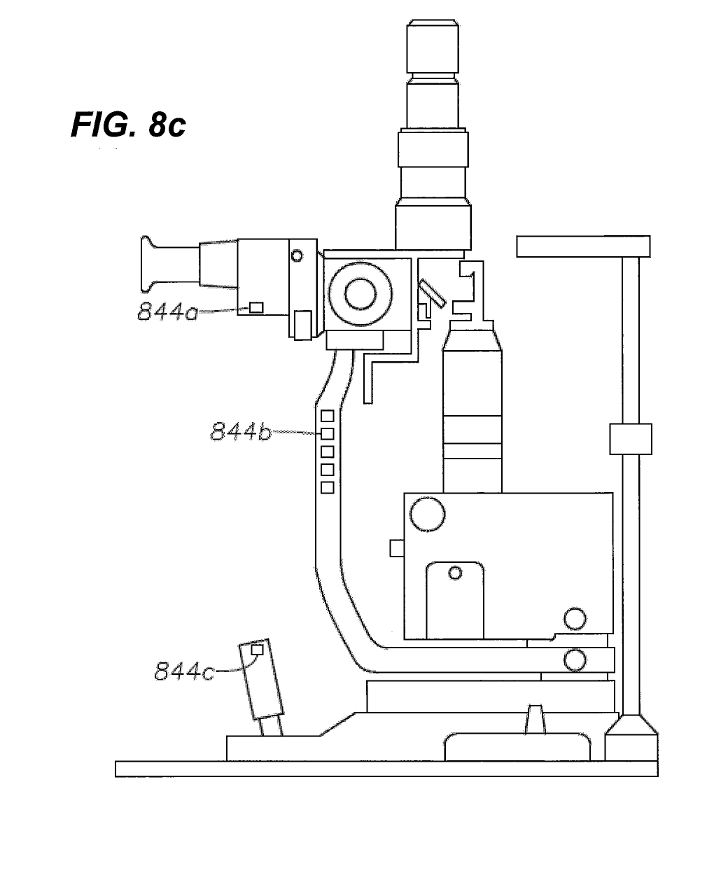

As seen in FIG. 6a, a footswitch 610a (e.g., for use with a laser console such as laser console 810 shown in FIG. 8a) may include a shroud 695 with indicators (e.g., indicators 691a-f). In some embodiments, the footswitch 610a may include a shroud 695 with a switch attached to an inner surface of the shroud 695 beside, below, or above a operator's foot. The operator may actuate the switch using an upward motion of his or her foot. In some embodiments, the laser may include a “stand-by/ready” switch 662 placed on a side wall of the shroud 695 such that a side motion of the operator's foot may actuate the switch 662. In some embodiments, laser firing switch 664 may be attached inside shroud 695. Other positions are also contemplated. Indicators 691e,f may indicate a switch status such as illuminating when the switch is pressed, illuminating in different colors for different respective modes corresponding to the switch pressed and/or how many times the switch was pressed, etc. In some embodiments, indicators 691a-d may indicate, for example, a firing sequence, which laser port is selected, a power level, system status, etc. Indicators 691e-f, for example, may be illuminated to indicate when the corresponding switch they are placed on is active and/or has been pressed.

During a laser surgery, an operator may move around the patient and/or position himself or herself in various different positions relative to a patient's eye. Consequently, the footswitch for firing the laser may also be moved around during a surgery. The operator may also use the footswitch shroud 695 to pick-up and move the footswitch (e.g., by using the shroud 695 as a sort of slipper). Because of this, a switch operable to place a laser in a ready condition from a stand-by condition and that is positioned above the operator's foot inside the shroud 695 may be actuated each time the operator lifts the footswitch resulting in possible undesired switching of the laser from standby to ready. In some embodiments, undesired switching may be prevented by incorporating sensors into the footswitch to detect the footswitch being lifted off the ground when being repositioned. Thus a switch actuation due to repositioning the footswitch and a switch actuation to affirmatively switch a laser from a stand-by to a ready condition or vice-versa may be differentiated using the incorporated sensors. Lifting sensors incorporated into the footswitch to detect such movement may include, for example, accelerometers, button switches on the bottom of the footswitch, ultrasound proximity sensors, optical sensors, a radio frequency signal modulation sensor, a radar sensor or any other such sensor operable to detect lifting of the surgical footswitch. The footswitch may also include sensors (e.g., positioned along the shroud 695) that can detect, for example, insertion of the operator's foot into the shroud 695 in preparation for use. The sensors may cause control signals to be generated, for example, that are operable to cause the laser to warm up in preparation for use. In this way, laser reliability can be increased while also decreasing lag times during, the surgery. Signals from the sensors may be used by the processors (e.g., processors 435/437) to determine appropriate configurations for illuminating indicators to relay information relative to the signals to an operator/staff. For example, indicators 693a and 693d may be illuminated one color to indicate when the shroud 695 is lifted off of the ground and a different color when the laser is switched to a ready condition. Indicators in different positions may also be illuminated to indicate status. For example, indicators 691a and 691d may be illuminated when the footswitch is lifted off of the ground and indicators 691b and 691c may be illuminated when the laser is switched to the ready condition. Other indicator configurations are also contemplated.

In some embodiments, the operator may independently control the stand-by/ready condition of the laser and fire the laser, from a single multifunction surgical footswitch. Indicators on the footswitch may convey information about the laser status, which port(s) are firing, the laser fire pattern, etc. to the operator. Dedicated mode switches and indicators may also be used on the laser surgical console. The operator may not need to use his or her hands or rely on an assistant to transition the laser from stand-by to ready, and vice versa, determine laser status, firing pattern, etc. during a surgery, freeing the operator to dedicate his or her attention to the surgical field.

FIG. 6b shows a diagram of an alternate embodiment of a footswitch 610b with a shroud 697 and a seventh arrangement of indicators. The footswitch 610b may include a body or housing 612 that includes a shroud 697 and a heel plate 616. Shroud 697 and heel plate 616 may be a single integrated assembly or separate units coupled together to form housing 612. All of these components may be made from any suitable material, such as stainless steel, titanium, or plastic. Embodiments may include a handle 618 that may be attached to housing 612. A first (laser stand-by/ready) switch 622 and a second (laser firing) switch 620 may be attached inside shroud 697. Laser firing switch 620 may be positioned forward of and on or near the same plane as heel plate 616, such that an operator inserting his or her foot into shroud 697 can press down on laser switch 620 while placing his or her heel on some portion of heel plate 616. In some embodiments, surgical footswitch laser stand-by/ready switch 622 may be attached inside shroud 697 such that it may be positioned above the ball/toes of an operator's foot and may be actuated by an upward motion of the operator's foot. When actuating a stand-by/ready switch 622 positioned this way, housing 612 may be maintained on a surface, such as a floor, by the pressure of an operator's heel pressing down on heel plate 616.

Laser firing switch 620 may include a press and hold type switch that may fire a single shot of varying duration or multiple shots, depending on the operator's configurable laser setting. Laser stand-by/ready switch 622 may be a single action button switch that may switch the laser mode from stand-by to ready (or vice-versa) upon pressing and release. However, either switch may be any other type switch as known to those having skill in the art that may perform the functions described herein.

Footswitch 610b may also include an interface 623, with one or more cable assemblies 624 to operably couple the footswitch 610b to a surgical console 428/laser 628 and operable to communicate control signals from footswitch 610b to console 428/laser 628. Surgical console 428 is operable to control laser 628, for example, to cause laser 628 to switch modes and/or to fire based on the control signals that are relayed from the footswitch 610b to the surgical console 428. In some embodiments, surgical console 428 may include control and/or processing circuitry for laser 628 (e.g., as part of processor 435/437), whether surgical console 428 is a separate enclosure or the same enclosure as that of laser 628. Surgical console 428 can be a console housing laser 628, including, for example, a multi-purpose console, such as a vitreo-retinal surgical console that includes laser 628, or a dedicated laser enclosure. In some embodiments, the surgical console may convey information about the laser 628 or other surgical parameters through indicators on the laser, footswitch, etc.

Another embodiment of footswitch 610b may include a communication interface 650, as shown in FIG. 6c, that is operable to establish a communication pathway (wired or wireless) between footswitch 610b and surgical console 428 to accomplish similar control signal transmission in a wireless manner. A wireless footswitch is disclosed in related U.S. patent application 60/667,290 filed Mar. 31, 2005, the entire contents of which are incorporated herein by reference. Surgical console 428 and laser 628 may be, for example, an EYELITE® photocoagulator manufactured by Alcon Laboratories, Inc. of Irvine, Calif.

The embodiment of FIG. 6b illustrates a laser stand-by/ready switch 622 attached inside shroud 697 and positioned above where the ball of an operator's foot will normally be when footswitch 610b is in use. However, stand-by/ready switch 622 can be positioned, for example, on an inner side of shroud 697, or next to laser firing switch 620 on the base of housing 612. The position of stand-by ready switch 622 may be changed to accommodate a given implementation. Further, embodiments of the footswitch 610b may include one or more additional switches attached to footswitch 610b and each operable to provide a control signal operable to control a function at surgical console 428 (e.g., adjust laser power, pulse duration, etc.).

Typically, the stand-by/ready transition of the laser 628 is initiated when the stand-by/ready switch 622 is released, not when it is engaged. One embodiment of the footswitch may include a stand-by/ready switch 622 of this type together with a lifting sensor assembly 630 placed, for example, on or beneath the footswitch 610b and operable to detect lifting of footswitch 610b. Such an embodiment may provide the ability to distinguish between an operator engaging stand-by/ready switch 622 to change the laser's status, and an operator lifting footswitch 610b to move it around (for example, when using footswitch 610b with the Alcon LIO System manufactured by Alcon Laboratories, Inc. of Irvine, Calif.).

In an embodiment incorporating a lifting sensor assembly 630, when an operator lifts footswitch 610b to move it, although he or she will engage stand-by/ready switch 622, the lifting sensor assembly 630 may detect the lifting of footswitch 610b from its supporting surface. Corresponding indicators (e.g., indicators 693a,d) may illuminate to indicate the footswitch 610b is being lifted and therefore, the stand-by/ready switch 622 is not active. In some embodiments, indicators 693b,c may illuminate when the footswitch 610b is detected on the ground and the laser is ready for firing. Lifting sensor assembly 630 may prevent the actuation (release) of stand-by/ready switch 622 from causing laser 628 to change modes when lifting sensor assembly 630 detects lifting of footswitch 610b from a supporting surface, such as a floor. Thus, after an operator lifts, moves and returns footswitch 610b to the supporting surface, stand-by/ready switch 622 may be released, but the release (actuation) of switch 622 may not result in a stand-by/ready transition of laser 628. Lifting sensor assembly 630 may not prevent a desired switching of the laser 628 stand-by/ready condition during normal operation because the pressure of the operator's heel on heel plate 616 may prevent lifting of footswitch 610b. Lifting sensor assembly 630 may include accelerometers, button switches on the bottom of the footswitch, pressure sensors, ultrasound proximity sensors, optical sensors, a radio frequency signal modulation sensor, a radar sensor or any other such sensor operable to detect lifting of the surgical footswitch.

Another embodiment may incorporate sensors, such as a foot sensor assembly 636, into, for example, shroud 697 of housing 612 to detect the presence of an operator's foot within the shroud 697. Foot sensor assembly 636 may detect the operator's foot and provide a control signal to console 428 operable, for example, to warm up laser 628 or otherwise prepare the laser surgical system for firing. Foot sensor assembly 636 may include, for example, ultrasound proximity sensors, a mechanical switch gate (e.g., a shroud entry door), an optical light gate (e.g., LED photodiode or laser photodiodes), radio frequency (“RF”) signal modulation sensors, radar sensor, accelerometers, an optical sensor or any such sensor operable to sense such movement. Embodiments of the footswitch may include a combination of such lift and/or foot presence sensors.

FIG. 6c is a functional diagram of an embodiment of the multifunction surgical footswitch incorporating a communication interface 650 (which may have wired or wireless communications) for communicating control signals for the various functions of the footswitch. In this embodiment, surgical footswitch 610b includes an input device 640, which may be, for example, a pedal, an encoder assembly 642 and a communication interface 650. This embodiment also includes two switches, a first switch 646 and a second switch 648, that operably couples to the mechanical input device 640. Encoder 642 may encode the control signals to be transmitted by communication interface 650 (which may be an interface for communications over connection 670 (which may include wired or wireless communications)) to surgical console 428. An embodiment may also include the operably coupled first switch 646, second switch 648 and mechanical input device 640 with a wired or wireless interface of FIG. 6b.

As shown in FIG. 6c, the surgical footswitch 610b may include wired or wireless communications (e.g., through pathway 670) and a progressive laser firing sequence. Input device 640 is analogous to the laser firing switch 620 of FIG. 6b in its laser firing function. Input device 640 may include a progressive actuation functionality. As shown in FIG. 6c, the footswitch may include a combination of switches and functions as described herein, and in particular the stand-by/ready switching functionality. Input device 640 may include a pedal, other mechanical input device, or a device that can provide the progressive action as described herein.

In operation, first switch 646 may be actuated and generate a first control signal as input device 640 orients past a first determined point. The first control signal may be operable, for example, to initialize surgical laser 628 within the surgical system. The first switch 646 may be activated, for example, when the input device 640 is initially depressed. The second switch 648 may produce a second control signal offset in time from the first control signal produced by the activation of first switch 646. For example, second switch 648 may be activated as pedal 640 nears the end of its angular range of motion (i.e., when the pedal 640 is fully depressed). The second control signal may direct the firing of surgical laser 628.

The trigger time between the activation of first switch 646 and second switch 648 may allow, for example, the stress on the laser 628 to be reduced. In such an implementation, the trigger time between the activation of the first switch 646 and the second switch 648 may allow the laser 628 to slowly warm up before firing. Note that the functionality of first switch 646 and second switch 648 may be incorporated within a single laser firing switch 620 described with reference to FIG. 6b. For example, referring back to FIG. 6b, stand-by/ready switch 622 may be depressed and released to place the laser 628 in a ready condition from a stand-by condition. Then, laser firing switch 620, which may incorporate the functions of first switch 646 and second switch 648, may ramp the laser 628 up to firing and then fire the laser 628 in the continuous movement of the operator's foot from initially depressing input device 640 (actuating first switch 646) to fully depressing input device 640 (actuating second switch 648).

In such an embodiment, laser firing switch 620 may include a pedal, such as pedal 640, operably coupled to a multi-position switch or switches having the functionality of first switch 646 and second switch 648. In one embodiment, the trigger time between the activation of the two switches 646 and 648 may be between about 100 ms and 300 ms.

The actual time may depend on the foot speed of the operator. This may allow laser 628 to be slowly ramped to power over the span of about 100 ms to about 300 ms (note that this is after the laser has already been placed in a ready condition from a stand-by condition). This may be useful for certain lasers that can not be turned on in less than 50 ms. The reduced stress associated with firing the laser in accordance with this embodiment may result in improved laser performance and reliability. Footswitch 610b may be physically coupled to the control circuits associated with initializing and firing laser 628, such as by a cable assembly 624 of FIG. 6b.

FIG. 7 illustrates a surgical console 428, according to an embodiment. In some embodiments, surgical console 428 may include a phacoemulsification console, a laser console, a vitrectomy console, etc. Surgical console 428 may also include indicators 701 that may be positioned for easy operator viewing. For example, indicators 701 are shown on a side of the surgical console 428. Other locations for the indicators 701 are also contemplated.

FIG. 8a illustrates a laser console 810 and a laser indirect opthalmoscope (LIO) 820, according to an embodiment. In some embodiments, laser console 810 may include two laser ports (left laser port 850a coupled to laser probe 830 and right laser port 850b coupled to LIO 820). Information presented through indicators (e.g., indicators 840) may include a currently programmed laser fire pattern for laser probe 830 (which may be an endo probe). The indicators 840 may blink (turn on/off) in the same pattern that the laser is programmed to fire (the energy of which may be delivered to a body part such as the eye through laser probe 830). The illuminated pattern may provide the operator a preview of the firing parameters that are currently set on the console 810. For example, the indicators 840 may blink with 200 ms on and 300 ms off. As another example, the indicators 840 may be continuously on to show a continuous fire setting. Indicators 840 may also display different colors to indicate a status of the laser/console (e.g., green for “Ready”, white for “Standby”, and red for “Error”). As another example, the indicators 840 may be red to indicate that a laser port is disabled, the laser is disabled, or some other hardware/software fault is disabling a component of the laser system. Red or a different color may also be used to indicate the laser system is exceeding a safe thermal load (e.g., if the laser probe 830 has been continuously firing for more than 2 seconds, the indicators 840 on the laser probe 830 may quickly blink red to warn the operator of the exceeded thermal load). Different color indicators may also be used to indicate a relative temperature of the laser engine. For example, as one or more indicators 840 are blinking to show a firing sequence, the indicators 840 may blink as green for low engine temperature, white for normal engine temperature, and red for high engine temperature. The indicators 840 may be pre-programmed (i.e., different colors may be assigned to different color indicators). The indicators 840 may also blink red, show red continuously, etc. when an emergency switch is pressed and the laser is shutdown or, for example, when an unidentified laser/probe is coupled to a port of the console 428. Other indicator configurations/colors are also contemplated.

In some embodiments, indicators 840 may also indicate interval time set for the laser including a time between treatment shots when the laser is applying treatment shots in repeat mode. For example, the indicators 840 may blink in the same pattern as the treatment shots or may blink at a relative speed (e.g., blink slowly to indicate the treatment shots are being applied at a low repeat timing interval, blink quickly to indicate the treatment shots are being applied at a fast repeat timing interval, blink once to indicate single shot treatment shot, illuminate continuously to indicate continuous wave (CW) firing mode, etc). The indicators 840 may also indicate a pulse pattern by blinking in the same timing as the current pulse pattern (e.g., one every 10, 20, 50, 100, 150, 200, 250, 300, 400, 500, 700, 1000, 1500, 2000 ms). The pulse patterns may also be assigned an integer indicator (e.g., pulse pattern #1, pulse pattern #2, etc.) and the indicators 840 may blink the number of the current integer indicator associated with the current pulse pattern (e.g., blink twice to indicate pulse pattern #2). Separate indicators (e.g., of the three indicators of indicators 840) may also be assigned to various pulse patterns (e.g., one indicator may illuminate to indicate pulse pattern #1, a separate indicator may illuminate for pulse pattern #2, etc). The indicators may illuminate and/or blink a predetermined color to indicate when a doctor protection filter is missing. For example, a switch on the system may be configured to detect the presence of a doctor filter and may trigger the system to provide information through the indicators 840 to signify that a doctor filter is missing.

FIG. 8b illustrates a side view of the LIO 820 with indicators 842, according to an embodiment. In some embodiments, the LIO 820 may include indicators 842 that are visible to the operator. These indicators 842 may also blink in the programmed firing pattern, display a console status, etc. FIG. 8c illustrates a slit-lamp with doctor filter (which may be used to calibrate/test the laser), according to an embodiment. Indicators, such as indicators 844a-c may display the status of various elements of the slit-lamp with doctor filter. Indicators such as indicators 842 and 844a-c may be used to indicate a status of an LIO illumination brightness and an aiming beam intensity brightness (e.g., the brighter the LIO illumination or aiming beam intensity setting, the more indicators may be illuminated). The indicator positions/colors may also be used to relay various parameters of a calibration procedure for the laser (e.g., exposure time, beam spot size, power level compared to displayed level, etc) and/or may relay information relative to a patient examination. These parameters may be relayed through various characteristics of the indicators (e.g., color) used to indicate how a detected value of the parameter relates to an expected value of the parameter (e.g., indicators may be blue to show the detected value is lower than normal (such as exposure time <10 ms, power level <13% of displayed value, etc.); indicators may be green to show the detected value is normal (such as exposure time approximately equal to 10 ms, power level approximately equal to displayed value, etc.); and indicators may be red to show the detected value is above normal (such as exposure time >10 ms, power level >13% of displayed value, etc.)). Indicators may also be illuminated next to lettering on the laser probe 830, slit-lamp, etc. indicating a parameter value (e.g., illuminated next to a respective label of “Low”, “Normal”, and “High”). Other indicators are also contemplated.

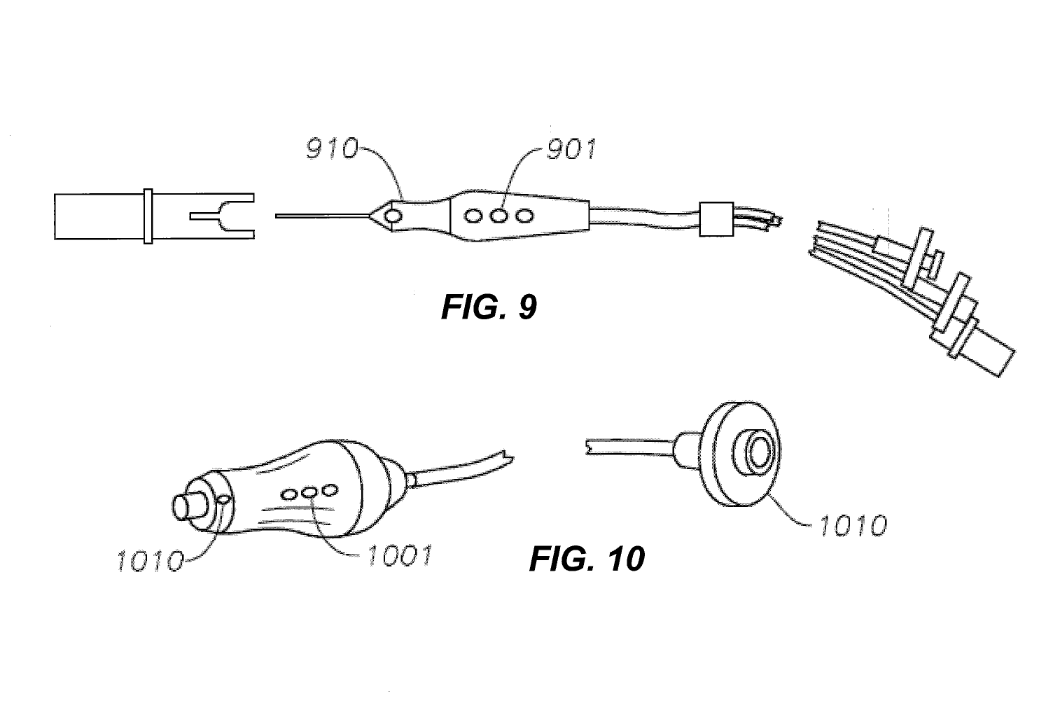

FIG. 9 illustrates a vitrectomy probe 910 with indicators 901, according to an embodiment. The indicators 901 on the vitrectomy probe 910 may also display a status of the vitrectomy system, a vitrectomy pattern to be or currently being implement, etc. Other uses of the indicators on the vitrectomy probe 910 are also contemplated.

FIG. 10 illustrates a pneumatic handle 1010 with indicators 1001, according to an embodiment. The indicators 1001 on the pneumatic handle 1010 may also display a status of the pneumatic system, a pneumatic pattern to be or currently being implemented, etc. Other uses of the indicators 1001 on the pneumatic handle 1010 are also contemplated.

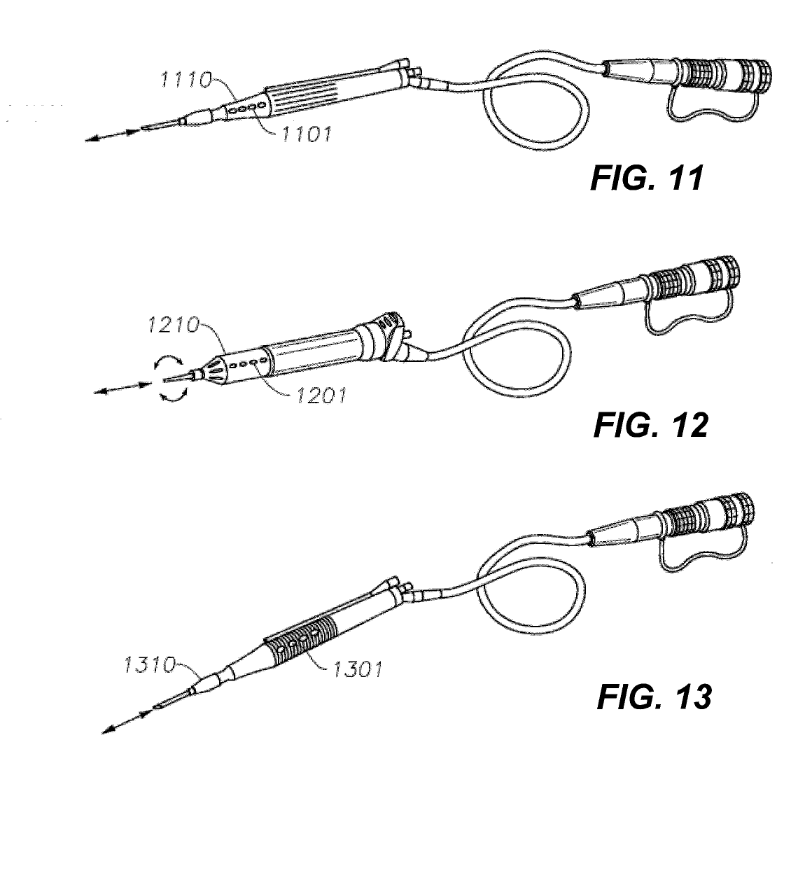

FIG. 11 illustrates a torsional handpiece 1110 with indicators 1101, according to an embodiment. The indicators 1101 on the torsional handpiece 1110 may also display a status of the torsional handpiece system, a torsional pattern to be or currently being implemented, ultrasound speed, error detected, etc. Other uses of the indicators 1101 on the torsional handpiece 1110 are also contemplated.

FIG. 12 illustrates another ultrasound handpiece 1210 with indicators 1201, according to an embodiment. FIG. 13 illustrates yet another ultrasound handpiece 1310 with indicators 1301, according to an embodiment. The indicators 1201/1301 on the ultrasound handpieces 1210/1310 may also display a status of the ultrasound handpiece system, an ultrasound pattern (e.g., pulse/burst pattern) to be or currently being implemented, ultrasound speed, error detected, etc. Other uses of the indicators 1201/1301 on the ultrasound handpiece 1210/1310 are also contemplated.

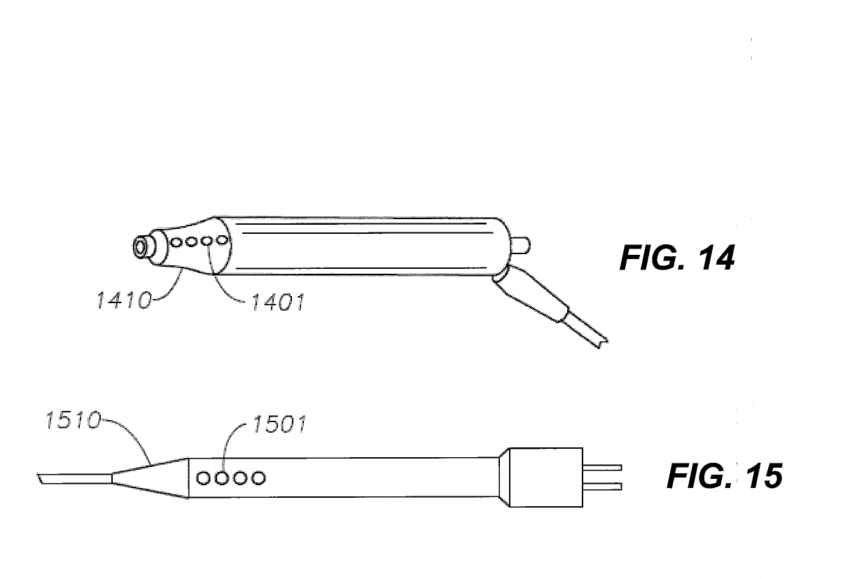

FIG. 14 illustrates a fragmentation handpiece 1410 with indicators 1401, according to an embodiment. The indicators 1401 on the fragmentation handpiece 1410 may also display a status of the fragmentation handpiece system, a fragmentation pattern to be or currently being implemented, etc. Other uses of the indicators 1401 on the fragmentation handpiece 1410 are also contemplated.

FIG. 15 illustrates a diathermy/coagulation handpiece 1510 with indicators 1501, according to an embodiment. The indicators 1501 on the diathermy/coagulation handpiece 1510 may also display a status of the diathermy/coagulation handpiece system, a diathermy/coagulation pattern to be or currently being implemented, etc. Other uses of the indicators 1501 on the diathermy/coagulation handpiece 1510 are also contemplated.

FIG. 16 illustrates a flowchart of an embodiment of a method for providing information about parameters, console status, etc. to an operator through the use of indicators. The elements provided in the flowchart are illustrative only. Elements may be omitted, additional elements may be added, and/or various elements may be performed in a different order than provided below.

At 1600, information relative to a procedure (e.g., information relative to a surgical device (such as a footswitch, handpiece, other adaptation, etc.), surgical console, surgical parameter, etc. used in a surgical procedure, patient examination, calibration, etc.) may be detected. For example, a information may include a status of a surgical console (e.g., “Ready”, “Stand-by”, “Error”, etc.), an active laser port (e.g., left side laser port, right side laser port, etc.), a currently programmed laser pattern (e.g., continuous, blinking with 200 ms “on” and 300 ms “off”, etc.), a configuration of a multifunction footswitch (e.g., which switches are active and what the switches control), etc. In some embodiments, the information may be detected by a processor processing the information from a memory on a surgical console. In some embodiments, the information may be detected by a sensor coupled to the console, footswitch, handpiece, etc. The information may also be entered by an operator (e.g., into the console, using switches on the footswitch or handpiece, etc).

At 1602, a configuration for one or more indicators may be determined to provide the information to an operator. For example, an indicator color (such as green for “Ready”, white for “Stand-by”, and red for “error”), pattern (e.g., a blinking pattern to match a preprogrammed laser firing sequence), position (e.g., on a left side of a footswitch to indicate a left side laser port is active or on a right side of a footswitch to indicate a right side laser port is active), etc. may be used to provide the information to the operator/staff. The configuration for the indicators may be determined using preprogrammed instructions stored on a memory accessible to a processor on the console and/or footpiece/handpiece (or other adaptation). In some embodiments, the configuration may be pre-programmed and/or may be provided by the operator before or during a surgical procedure (e.g., by downloading code onto the console, entering parameters into a graphical user interface displayed by the console, etc). Other methods for determining the configuration are also contemplated.

At 1604, a control signal based on the determined configuration may be generated. In some embodiments, the system (e.g., processor 435 and/or 437 as seen in FIG. 4c) may provide a control signal.

At 1606, the control signal may be communicated to at least one indicator. In some embodiments, the control signal may be sent through communication interfaces (e.g., communication interface 424/430) between a console and/or footswitch/handpiece (or other adaptation)). In some embodiments, the control signal may be determined locally (e.g., at processor 435) and/or may be provided directly to the indicator in the form of power to illuminate the indicator.

At 1608, at least one indicator (e.g., on a footswitch, handpiece, or other adaptation) may be illuminated according to the communicated control signal. In some embodiments, the control signal may be a power signal to power the indicator. In some embodiments, the control signal may direct a control circuit coupled to one or more indicators to illuminate a specified indicator. Other control signal/indicator illumination associations are also contemplated.

While several embodiments are provided herein, it should be understood that other embodiments are also possible in other forms or variations thereof without departing from the spirit of the invention. The embodiments described herein are therefore considered to be illustrative in all respects and not restrictive, the scope of the invention being indicated by the appended claims. As may be used herein, the terms “substantially” and “approximately” provide an industry-accepted tolerance for their corresponding term and/or relativity between items. Such an industry-accepted tolerance ranges from less than one percent to fifty percent and corresponds to, but is not limited to, component values, integrated circuit process variations, temperature variations, rise and fall times, and/or thermal noise. Such relativity between items ranges from a difference of a few percent to magnitude differences. As may also be used herein, the term(s) “coupled to”, “operably coupled” and/or “coupling” include direct coupling between items and/or indirect coupling between items via an intervening item (e.g., an item includes, but is not limited to, a component, an element, a circuit, and/or a module). As may further be used herein, inferred coupling (i.e., where one element is coupled to another element by inference) includes direct and indirect coupling between two items in the same manner as “coupled to”. As may even further be used herein, the term “operable to” indicates that an item includes one or more of power connections, input(s), output(s), etc., to perform one or more its corresponding functions and may further include inferred coupling to one or more other items. As may still further be used herein, the term “associated with”, includes direct and/or indirect coupling of separate items and/or one item being embedded within another item. As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Various modifications may be made to the presented embodiments by a person of ordinary skill in the art. Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. An apparatus, comprising:
a surgical device, external to a surgical console, configured to be physically engaged by an operator during a surgical procedure;
an interface coupled to the surgical device and configured to communicate with a surgical console, wherein the interface is configured to receive information from the surgical console; and
an indicator on the device and configured to be illuminated to provide at least part of the information received from the surgical console to the operator.

2. The apparatus of claim 1, wherein the surgical procedure is an ophthalmic surgical procedure.

3. The apparatus of claim 1, wherein the device comprises a surgical footswitch or surgical handpiece.

4. The apparatus of claim 1, wherein the information is associated with a status of the surgical console and wherein the indicator is illuminated in different colors to indicate different console statuses.

5. The apparatus of claim 1,
wherein the surgical device comprises a footswitch;
wherein the surgical console comprises a left laser port and a right laser port;
wherein the indicator comprises a first indicator on a left side of a footswitch and a second indicator on a right side of the footswitch;
wherein the first indicator is configured to be illuminated when the left laser port is active and wherein the second indicator is configured to be illuminated when the right laser port is active.

6. The apparatus of claim 1, wherein the surgical console is coupled to a laser and wherein the indicator is configured to be illuminated in a timewise pattern that corresponds to a firing pattern of the laser.

7. The apparatus of claim 1, wherein the device is a multi-function footswitch and wherein illumination of the indicator is associated with a current configuration of the multi-function footswitch.

8. An apparatus, comprising:
a surgical device, communicatively coupled to a surgical console, configured to be physically engaged by an operator; and
at least one indicator on the surgical device configured to be illuminated in at least three different configurations to provide information relative to a characteristic of the surgical device or surgical console to the operator.

9. The apparatus of claim 8, wherein the at least three different configurations comprise at least three different blink patterns.

10. The apparatus of claim 8, wherein the at least one indicator comprises a plurality of indicators and wherein the at least three different configurations comprise three different illumination patterns of the plurality of indicators.

11. The apparatus of claim 8, wherein the surgical device is configured to be physically engaged by the operator during an ophthalmic surgical procedure and wherein the provided information is relative to a characteristic of the surgical device or surgical console used in the ophthalmic surgical procedure.

12. The apparatus of claim 8, wherein the device comprises a surgical footswitch or a surgical handpiece.

13. The apparatus of claim 8, wherein the information is associated with a status of the surgical console and wherein the indicator is illuminated in different colors to indicate different console statuses.

14. The apparatus of claim 8,
wherein the device comprises a footswitch;
wherein the surgical console comprises a left laser port and a right laser port;
wherein the indicator comprises a first indicator on a left side of a footswitch and a second indicator on a right side of the footswitch; and
wherein the first indicator is configured to be illuminated when the left laser port is active and wherein the second indicator is configured to be illuminated when the right laser port is active.

15. The apparatus of claim 8, wherein the surgical console is coupled to a laser and wherein the indicator is configured to be illuminated in a timewise pattern that corresponds to a firing pattern of the laser.

16. The apparatus of claim 8, wherein the device is a multi-function footswitch and wherein illumination of the indicator is associated with a current configuration of the multi-function footswitch.
